(19) United States
(12) Patent Application Publication
(10) Pub. No.: US 2016/0317594 A1
(43) Pub. Date: Nov. 3, 2016

(54) METHOD FOR BREAKING SPORODERM OF GANODERMA SPORE POWDER AND PRODUCTS OBTAINED BY THE SAME

(71) Applicant: Yuming Huang, Baiyun Distric (CN)

(72) Inventors: Yuming Huang, Guangzhou (CN); Jian Teng, Changsha (CN); Xu Li, Changsha (CN)

(21) Appl. No.: 14/945,439

(22) Filed: Nov. 19, 2015

(30) Foreign Application Priority Data
Apr. 30, 2015 (CN) 201510217208.3

(51) Int. Cl. A61K 36/074; A23L 2/52

(52) U.S. Cl. CPC A61K 36/074; A23L 1/28; A23L 1/30; A23L 2/52; A23V 2002/00

(57) ABSTRACT

The present invention provides a method for breaking sporoderm of Ganoderma spore powder, and food and beverage products obtained by the same. The sporoderm-breaking method includes the steps of: putting 5 to 10 parts by weight of Ganoderma spore powder into 60 to 100 parts by volume of water; stirring fully to form a mixture; performing a sporoderm-breaking treatment on the mixture to obtain a sporoderm-broken spore powder solution; optionally, filtering the sporoderm-broken spore powder solution to obtain a plurality of filtrates; and pooling and drying the filtrates to obtain a dried Ganoderma spore powder extract. The sporoderm-breaking treatment comprises slowly heating the mixture to a temperature ranging between 63 and 100° C.; and maintaining at the temperature for 15 to 60 min. The sporoderm-breaking method of the present invention can significantly improve sporoderm-breaking rate of Ganoderma spore powder, preserve the active ingredients in raw materials of Ganoderma spore powder.

### FIELD OF THE INVENTION

The present invention relates to a processing technology of ganoderma spore powder, in particularly relates to a sporoderm-breaking method capable of fully releasing active ingredients of Ganoderma spores and products obtained by the same.

### BACKGROUND OF THE INVENTION

Since ancient times, the Chinese traditional medicine has confirmed that Ganoderma lucidum, also known as the entire plant of a polyporaceae plant, has the effects of nourishing and building up the body and supporting health energy; and the modern pharmacological and clinical studies have also proved the pharmacologic effects of Ganoderma lucidum. Therefore, Ganoderma lucidum has been formally recorded as a legal traditional Chinese herbal medicine in Chinese Pharmacopoeia. Meanwhile, Ganoderma lucidum is also a state-approved new resource food; that is, Ganoderma lucidum may be used as food and medicine.

Ganoderma spore powder is extremely small ovoid germ cells ejected from Ganoderma lamella when Ganoderma lucidum is in a mature growth stage. Ganoderma spore powder aggregates all genetic materials of Ganoderma lucidum and nutrient substances required for germination of a new life, and is rich in glycopeptides, sterols, triterpenes, nucleosides, alkaloids, multiple microelements and other physiologically active ingredients. The modern pharmaceutical studies have showed that Ganoderma spore powder has a higher medicinal value than the sporocarp and mycelium of Ganoderma lucidum. However, as the Ganoderma spore powder is wrapped by one layer of sporoderm which is composed of hard chitin cellulose and difficult to be decomposed by the digestive juice. The Ganoderma spore powder with a sporoderm is difficult to be digested and absorbed by the human body if it is directly ingested, so it is required to perform a sporoderm-breaking treatment on the sporoderm such that the Ganoderma spore powder may be absorbed and utilized by the human body to the largest extent. Scientific experiments have proved that only 10% to 20% of active ingredients may be absorbed by the human body when sporoderm-intact Ganoderma spore powder is eaten, while the absorption rate of the active ingredients may reach 90% or above after breakage of the sporoderm.

At present, the sporoderm-breaking technologies for Ganoderma spore powder include a physical method, a chemical method, and a biological enzymolysis method, etc., among which the physical method is applied most commonly, as the chemical method, which mainly breaks the sporoderm by soaking in alkali solutions, and the biological enzymolysis method, which decomposes the sporoderm by a biological enzyme, both have lower sporoderm-breaking rates than the physical method and are more likely to introduce impurities into and/or destroy nutrient components of sporoderm-broken Ganoderma spore powder. The approach adopted by the physical method is usually to perform mechanical grinding, high-pressure gas smashing, high-frequency oscillation or other treatments on sterilized and dried Ganoderma spore powder. However, the local high temperature generated in the treatments is likely to result in oxidative deterioration of the active ingredients in the Ganoderma spore powder, and chipping of the Ganoderma spore powder that leads to secondary pollution is also likely. All the methods involve high cost and specific industrial equipments; additionally, the sporoderm-broken Ganoderma spore powder is likely to oxidized during storage and thus generates a rancid smell that greatly reduces the efficacy of the Ganoderma spore powder. Therefore, the sporoderm-broken Ganoderma spore powder is unsustainable for long-term storage.

In order to solve the aforementioned deficiencies existing in the prior art, how to develop a sporoderm-breaking method that can conveniently, time-effectively and efficiently break the sporoderm of long-term stored and sporoderm-intact Ganoderma spore powder and would not introduce any new impurities while fully preserving the nutrient components has become an urgent technical challenge for those skilled in the art.

### SUMMARY OF THE INVENTION

The present invention provides a simple and fast sporoderm-breaking method for ganoderma spore powder, which has high sporoderm-breaing rate and would not introduce any new impurities while fully preserving the nutrient components. Products obtained by the same is also provided.

To solve the technical challenge, the present invention employs the following technical solutions.

A method for breaking sporoderm of Ganoderma spore powder includes the following steps:

putting 5 to 10 parts by weight of Ganoderma spore powder into 60 to 100 parts by volume of water; stirring fully to form a mixture; performing a sporoderm-breaking treatment on the mixture for 1 to 3 times to obtain a sporoderm-broken spore powder solution;

optionally, filtering the sporoderm-broken spore powder solution to obtain a plurality of filtrates; and pooling and drying the filtrates to obtain a dried Ganoderma spore powder extract,

wherein the sporoderm-breaking treatment includes slowly heating the mixture to a temperature ranging between 63 and 100° C.; and maintaining at the temperature for 15 to 60 min.

In an embodiment of the present invention, the sporoderm-breaking treatment further includes: slowly heating the mixture to a temperature ranging between 96 and 100° C.; and maintaining the temperature for 15 to 20 min.

In an embodiment of the present invention, the sporoderm-breaking treatment further includes: slowly heating the mixture to 70° C., 75° C., 80° C., 85° C., 90° C. or 95° C.; and maintaining at one of the temperatures for 20 min, 25 min, 30 min, 35 min, 40 min, 45 min, 50 min or 55 min.

In an embodiment of the present invention, the method for breaking sporoderm of Ganoderma spore powder preferably includes the following steps:

putting 5 parts by weight of the Ganoderma spore powder into 100 parts by volume of distilled water; stirring fully to form a mixture; slowly heating the mixture to 63° C. and maintaining at 63° C. for 45 min to obtain the sporoderm-broken spore powder solution; filtering the sporoderm-broken spore powder solution to obtain the plurality of filtrates, and pooling and drying the filtrates to obtain the dried ganoderma spore powder extract.

In an embodiment of the present invention, the method for breaking sporoderm of ganoderma spore powder preferably includes the following steps:

putting 7 parts by weight of the Gganoderma spore powder into 70 parts by volume of distilled water; stirring fully to form the mixture; slowly heating the mixture to 96° C. and maintaining at 96° C. for 20 min to obtain a primary sporoderm-broken spore powder solution; filtering the primary sporoderm-broken spore powder solution to obtain a primary filtrate and a primary filtration residue;

adding 70 parts by volume of distilled water into the primary filtration residue; stirring fully; slowly heating to 100° C. and maintaining at 100° C. for 20 min to obtain a secondary sporoderm-broken spore powder solution; filtering the secondary sporoderm-broken spore powder solution to obtain a secondary filtrate; pooling the secondary filtrate and the primary filtrate; and

optionally, drying the pooled secondary filtrate and primary filtrate to obtain the dried Ganoderma spore powder extract.

In an embodiment of the present invention, the method for breaking sporoderm of Ganoderma spore powder preferably includes the following steps:

putting 10 parts by weight of the Ganoderma spore powder into 60 parts by volume of distilled water; stirring fully to form the mixture, slowly heating the mixture to 78° C. and maintaining at 78° C. for 60 min to obtain a primary sporoderm-broken spore powder solution; filtering the primary sporoderm-broken spore powder solution to obtain a primary filtrate and a primary filtration residue;

adding 70 parts by volume of distilled water into the primary filtration residue; stirring fully; slowly heating to 90° C. and maintaining at 90° C. for 25 min to obtain a secondary sporoderm-broken spore powder solution; filtering the secondary sporoderm-broken spore powder solution to obtain a secondary filtrate, pooling the secondary filtrate and the primary filtrate;

optionally, drying the pooled secondary filtrate and primary filtrate to obtain the dried Ganoderma spore powder extract.

In an embodiment of the present invention, the method for breaking sporoderm of Ganoderma spore powder preferably includes the following steps:

putting 5 parts by weight of the Ganoderma spore powder into 80 parts by volume of distilled water; stirring fully to form the mixture, slowly heating the mixture to 100° C. and maintaining at 100° C. for 15 min to obtain a primary sporoderm-broken spore powder solution; filtering the primary sporoderm-broken spore powder solution to obtain a primary filtrate and a primary filtration residue;

adding 80 parts by volume of distilled water into the primary filtration residue; stirring fully; slowly heating to 100° C. and maintaining at 100° C. for 15 min to obtain a secondary sporoderm-broken spore powder solution; filtering the secondary sporoderm-broken spore powder solution to obtain a secondary filtrate and a secondary filtration residue;

adding 80 parts by volume of distilled water into the secondary filtration residue; stirring fully; slowly heating to 100° C. and maintaining at 100° C. for 15 min to obtain a tertiary sporoderm-broken spore powder solution; filtering the tertiary sporoderm-broken spore powder solution to obtain a tertiary filtrate; pooling the primary filtrate, the secondary filtrate and the tertiary filtrate; and

optionally, drying the pooled primary filtrate, secondary filtrate, and tertiary filtrate to obtain a dried Ganoderma spore powder extract.

Further, the Ganoderma spore powder is Ganoderma spore powder not having been subjected to a sporoderm breaking treatment.

A dried Ganoderma spore powder extract obtained by the method for breaking sporoderm of Ganoderma spore powder of the present invention is also provided.

A Ganoderma spore powder-containing food or beverage directly obtained by the method for breaking sporoderm of Ganoderma spore powder is also provided.

A food or beverage containing at least the dried Ganoderma spore powder extract is also provided.

A method for preparing the food or beverage is also provided. The method includes the following steps: putting Ganoderma spore powder and raw materials for a food or a beverage into 60 to 100 parts by volume of water; stirring fully; slowly heating the mixture to a temperature ranging between 63 and 100° C. and maintaining at the temperature for 15 to 60 min.

Preferably, the food includes, but is not limited to: porridge, steamed bun, cake, bread, cookie or other staple foods, and desserts.

Preferably, the beverage includes, but is not limited to: carbonated beverage, fruit-vegetable juice beverage, tea beverage, milk beverage, coffee beverage and alcoholic beverage.

In the present invention, the units of the part by weight/part by volume are g/mL.

As compared with existing sporoderm-breaking techniques for Ganoderma spore powder, the method for breaking sporoderm of Ganoderma spore powder of the present invention has the following advantages:

The method for breaking sporoderm of Ganoderma spore powder of the present invention utilizes water decoction to decoct raw materials of Ganoderma spore powder, and significantly improves the sporoderm-breaking rate of Ganoderma spore powder and thus the contents of active ingredients such as ganoderans, triterpenes and sterols in the decoction solution. Therefore, the method for breaking sporoderm of Ganoderma spore powder of the present invention can preserve the active ingredients in raw materials of Ganoderma spore powder to the largest extent. As compared with existing sporoderm-breaking techniques for ganoderma spore powder, the method for breaking sporoderm of Ganoderma spore powder of the present invention has the advantages of simple operation and easy implementation, as alkali treatment and biological enzyme fermentation, as well as, various sporoderm-breaking equipments are not required. Meanwhile, as the sporoderm-breaking method of the present invention only uses water, no new impurities would not be introduced, thus ensuring the high purity of the Ganoderma spore powder. In addition, the method for breaking sporoderm of Ganoderma spore powder of the present invention helps sustain long-term storage of Ganoderma spore powder. The active ingredients of Ganoderma spore powder can be released in as short as 15 to 20 min when decocted.

### BRIEF DESCRIPTION OF THE DRAWINGS

The present invention will become more readily apparent to those ordinarily skilled in the art after reviewing the following detailed description and accompanying drawings, in which:

FIG. 1 shows an electron microscopic image of Ganoderma spores after 30 min of decoction according to an embodiment of the present invention;

FIG. 2 shows an electron microscopic image of Ganoderma spores after 10 min of decoction according to an embodiment of the present invention;

FIG. 3 shows an electron microscopic image of Ganoderma spores after 20 min of decoction according to an embodiment of the present invention;

FIG. 4 shows an electron microscopic image of Ganoderma spores after 20 min of microwaving according to an embodiment of the present invention;

FIG. 5 shows an optical microscopic image (400× magnification) of sporoderm-intact Ganoderma spores according to an embodiment of the present invention; and

FIG. 6 shows an optical microscopic image (400× magnification) of sporoderm-broken Ganoderma spores according to an embodiment of the present invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

### EXPERIMENTAL EXAMPLE 1

### Study on Water Temperature

1.1 Objective

By adjusting the temperature of distilled water, the influences of the method for breaking sporoderm of Ganoderma spore powder of the present invention on the morphology, smell, pH, and sporoderm-breaking rate of Ganoderma spore powder solution and on the contents of ganoderans, triterpenes, and sterols in the Ganoderma spore powder solution under different water temperature conditions assessed.

1.2 Experimental Method

4 parts, with each part containing 5.0 g, of raw materials of Ganoderma spore powder are weighed and placed into four 250 ml beakers respectively. 100 ml of distilled water of different temperatures is then added into each of the beakers, and is stirred fully. Morphology, smell and pH of the Ganoderma spore powder solutions are observed and recorded; and the contents of ganoderans, triterpenes, and sterols in the solutions and the sporoderm-breaking rate are measured. The results are shown in Table 1.

Method for measuring the content of ganoderans is performed as follows:

(1) Preparation of a control solution: anhydrous glucose standard is weighed precisely and added with water to obtain a solution containing 0.12 mg of glucose every 1 ml of the solution.

(2) Establishment of a standard curve: 0.2 ml, 0.4 ml, 0.6 ml, 0.8 ml, 1.0 ml and 1.2 ml of the control solution are precisely measured and each placed into a 10 ml test tube with a stopper. Water was added to the tubes until reaching 2.0 ml. 6 ml of anthrone sulfate solution (0.1 g of anthrone fully dissolved in 100 ml of sulfuric solution by shaking) is then quickly and precisely added into the solutions; immediately followed by mixing and letting stand for 15 min. Thereafter, the solution is immediately placed in an ice bath for cooling for 15 min. A solution containing no glucose obtained by the aforementioned preparation method is used as a blank. In accordance with UV-Vis spectrophotometric methods (appendix VA, Chinese Pharmacopoeia, volume 1, 2010), UV-Vis absorbency of the samples at the wavelength of 625 nm is measured. Therefrom, a standard curve is drawn with absorbency as the y-axis and concentration as the x-axis.

(3) Preparation of a test solution: The aforementioned Ganoderma spore powder solution is filtered; the filtrate is evaporated to dryness in a water bath. The residue is dissolved with 5 ml of water, and 75 ml of ethanol was slowly dripped into the solution while stirring. The mixture is fully mixed and let stand for 12 hours at 4° C., followed by centrifugation. The supernatant is discarded; the precipitate is dissolved with hot water and transferred into a 50 ml flask; the solution is let stand to cool, and is added and fully mixed with water to a total volume of 50 ml. A portion of the solution is centrifuged, and 3 ml of supernatant is precisely measured and placed into a 25 ml flask. The solution is added and fully mixed with water to a total volume of 25 ml. 2 ml of the solution is precisely measured and transferred into a 10 ml test tube; 6 ml of anthrone sulfate solution (0.1 g of anthrone fully dissolved in 100 ml of sulfuric acid by shaking) was immediately mixed with the solution in the test tube. Finally, the test solution is obtained by letting the solution stand for 15 min and cooled immediately in an ice bath for 15 min.

(4) Determination of the content of ganoderans: 2 ml of water is precisely measured and placed into a 10 ml test tube with a stopper. 6 ml of anthrone sulfate solution (0.1 g of anthrone fully dissolved in 100 ml of sulfuric acid by shaking) was quickly added into the tube and fully mixed by shaking. The solution is let stand for 15 min, and immediately placed in an ice bath for cooling for 15 min, resulting in a blank control solution. By using the blank control solution as a blank control, the absorbency of the test solution at the wavelength of 625 nm is measured in accordance with UV-Vis spectrophotometric methods. The content of anhydrous glucose, therefore the content of ganoderans, in the test solution is determined according to the standard curve.

Method for measuring the contents of triterpenes and sterols is performed as follows:

(1) Preparation of a control solution: oleanolic acid standard is weighed precisely and added with methanol to obtain a solution containing 0.2 mg of oleanolic acid every 1 ml of the solution.

(2) Establishment of a standard curve: 0.1 ml, 0.2 ml, 0.3 ml, 0.4 ml and 0.5 ml of the control solution are precisely measured, and each placed into a 15 ml test tube with a stopper. The control solutions are let evaporated and cooled, and are precisely added with 0.2 ml of freshly prepared vanillic aldehyde glacial acetic acid solution (0.5 g of vanillic aldehyde dissolved in 100 ml of glacial acetic acid) and 0.8 ml of perchloric acid. The mixtures are fully mixed by shaking, heated for 15 min in a water bath at 70° C., and immediately placed in an ice bath for cooling for 5 min. The solution is then precisely added with 4 ml of ethyl acetate and fully mixed by shaking. A corresponding reagent is used as a blank control. In accordance with UV-Vis spectrophotometric methods (appendix VA, Chinese Pharmacopoeia, volume 1, 2010), absorbency of the samples at the wavelength of 546 nm is measured. Therefrom, a standard curve is drawn with absorbency as the y-axis and concentration as the x-axis.

(3) Preparation of a test solution: the aforementioned Ganoderma spore powder solution is filtered; the filtrate is placed into a 100 ml flask. A proper amount of distilled water is used to rinse through the filter and the filtration residue separately, and pooled into the flask. The flask is added ethanol to a total volume of 100 ml and the solution is fully mixed by shaking. 0.2 ml of the solution is precisely measured, placed into a 15 ml test tube with a stopper, evaporated to dryness, let stand to cool, and precisely added with 0.2 ml of freshly prepared vanillic aldehyde glacial acetic acid solution (0.5 g of vanillic aldehyde dissolved in 10 ml of glacial acetic acid) and 0.8 ml of perchloric acid. The mixture is fully mixed by shaking, heated for 15 min in a water bath at 70° C., immediately placed in an ice bath for cooling for 5 min, and precisely added and fully mixed with 4 ml of ethyl acetate to obtain the test solution.

(4) Determination of the contents of triterpenes and sterols: 0.2 ml of water is precisely measured and placed into a 15 ml test tube with a stopper. The tube is evaporated to dryness, let stand to cool, and precisely added 0.2 ml of freshly prepared vanillic aldehyde glacial acetic acid solution (0.5 g of vanillic aldehyde dissolved in 10 ml of glacial acetic acid) and 0.8 ml of perchloric acid. The mixture is fully mixed by shaking, heated for 15 min in a water bath at 70° C., immediately placed in an ice bath for cooling for 5 min, and precisely added 4 ml of ethyl acetate to obtain a black control solution. By using the blank control solution as a blank control, the absorbency of the test solution at the wavelength of 546 nm is measured in accordance with UV-Vis spectrophotometric methods. The content of oleanolic acid in the test solution, therefore the contents of triterpenes and sterols, are determined according to the standard curve.

In accordance with the method for measuring sporoderm-breaking rate of sporoderm-broken Ganoderma spore powder as specified in Appendix A of Harvesting and Processing Technical Specifications of Ganoderma Spore Powder of State Standard GB/T 29344-2012 of the People's Republic of China, the sporoderm-breaking rate of the aforementioned Ganoderma spore powder solutions are measured.

Table 1 shows the influences of water temperature on the morphology, smell and pH of Ganoderma spore powder solution, and on the contents of ganoderans, triterpenes and sterols, and the sporoderm-breaking rate in the Ganoderma spore powder solution.

| | | | | | | Contents of |
|---|---|---|---|---|---|---|
| Item | | | | Sporoderm- | Content | triterpenes |
| Water | | | | breaking | of | and |
| temperature | Morphology | Smell | pH | rate | ganoderans | sterols |
| 16° C. | Brown | Slightly | 6.2 | — | 0.12% | 0.18% |
| | suspension, | sour | | | | |
| | no adherence to | | | | | |
| | flask wall | | | | | |
| 63° C. | Brown | Slightly | 6.3 | 4% | 0.17% | 0.23% |
| | suspension, | sour | | | | |
| | no adherence to | | | | | |
| | flask wall | | | | | |
| 78° C. | Brown | Slightly | 6.2 | 11% | 0.19% | 0.31% |
| | suspension, | sour | | | | |
| | no adherence to | | | | | |
| | flask wall | | | | | |
| 96° C. | Brown | Slightly | 6.1 | 27% | 0.23% | 0.38% |
| | suspension, | sour | | | | |
| | no adherence to | | | | | |
| | flask wall | | | | | |

Table 1 shows that with increasing water temperature the sporoderm-breaking rate of Ganoderma spore powder significantly increases, and the contents of ganoderans, triterpenes, and sterols in the Ganoderma spore powder solution also increase. The results indicate that elevated temperature would facilitate sporoderm breaking of Ganoderma spore powder and thus releasing of the active ingredients in the Ganoderma spore powder.

### EXPERIMENTAL EXAMPLE 2

### Study on Decoction Time

2.1 Objective

By adjusting the duration of water decoction, the influences of the method for breaking sporoderm of Ganoderma spore powder of the present invention on the morphology, smell, pH, and sporoderm-breaking rate of Ganoderma spore powder solution and on the contents of ganoderans, triterpenes, and sterols in the Ganoderma spore powder solution under different durations of decoction are assessed.

2.2 Experimental Method

6 parts, with each part containing 5.0 g, of raw materials of Ganoderma spore powder are weighed and placed into six 250 ml beakers respectively. 100 ml of distilled water is then added into each beaker, and the mixtures are stirred fully and heated to slightly boiling. Morphology, smell and pH of the Ganoderma spore powder solutions after different lengths of boiling are observed and recorded; and the contents of ganoderans, triterpenes and sterol in the solutions and the sporoderm-breaking rate are measured. The methods for determining the contents of ganoderans, triterpenes, and sterol and the sporoderm-breaking rate are performed according to Experimental example 1. The results are shown in Table 2.

Table 2 shows the influences of decoction duration on the morphology, smell and pH of Ganoderma spore powder solution, and on the contents of ganoderans, triterpenes, and sterols, and the sporoderm-broking rate in the Ganoderma spore powder solution.

| | | | | | | Contents of |
|---|---|---|---|---|---|---|
| Item | | | | Sporoderm- | Content | triterpenes |
| Decoction | | | | breaking | of | and |
| duration | Morphology | Smell | pH | rate | ganoderans | sterols |
| 0 min | Brown | Slightly | 5.6 | 67% | 0.31% | 0.47% |
| | suspension, | sour | | | | |
| | no adherence to | | | | | |
| | flask wall | | | | | |
| 5 min | Brown | Slightly | 5.3 | 72% | 0.88% | 0.66% |
| | suspension, | sour | | | | |
| | no adherence to | | | | | |
| | flask wall | | | | | |
| 10 min | Brown | Slightly | 4.7 | 92% | 1.31% | 0.91% |
| | suspension, | sour | | | | |
| | no adherence to | | | | | |
| | flask wall | | | | | |
| 15 min | Dark brown | Obviously | 4.2 | 95% | 1.57% | 1.22% |
| | suspension, | sour | | | | |
| | no adherence to | | | | | |
| | flask wall | | | | | |
| 20 min | Dark brown | Obviously | 4.3 | 95% | 1.56% | 1.28% |
| | suspension, | sour | | | | |
| | no adherence to | | | | | |
| | flaks wall | | | | | |
| 25 min | Dark brown | Obviously | 4.3 | 96% | 1.59% | 1.27% |
| | suspension, | sour | | | | |
| | no adherence to | | | | | |
| | flask wall | | | | | |

Table 2 shows that with increasing duration of water decoction the sporoderm-breaking rate of Ganoderma spore powder significantly increases, and the contents of ganoderans, triterpenes, and sterols in the Ganoderma spore powder solution also increase. The results indicate that extended decoction would facilitate sporoderm breaking of Ganoderma spore powder and thus releasing of the active ingredients in the Ganoderma spore powder. However, after decoction for 15 min, the sporoderm-breaking rate of Ganoderma spore powder, the contents of ganoderans, triterpenes, and sterols do not increase significantly; therefore, the preferred decoction duration of the present invention is 15 to 20 min. In this way, high sporoderm-breaking rate of Ganoderma spore powder and high contents of ganoderans, triterpenes and sterol are ensured, thus ensuring the quality of the sporoderm-broken Ganoderma spore powder.

### EXPERIMENTAL EXAMPLE 3

### Electron Microscopic Analysis of Poroderm-Breaking of Ganoderma Spore

Ganoderma spore solution is suction-filtered; the filtration residues are dried and subjected to electron microscopic analyses to observe the morphologies of the spores.

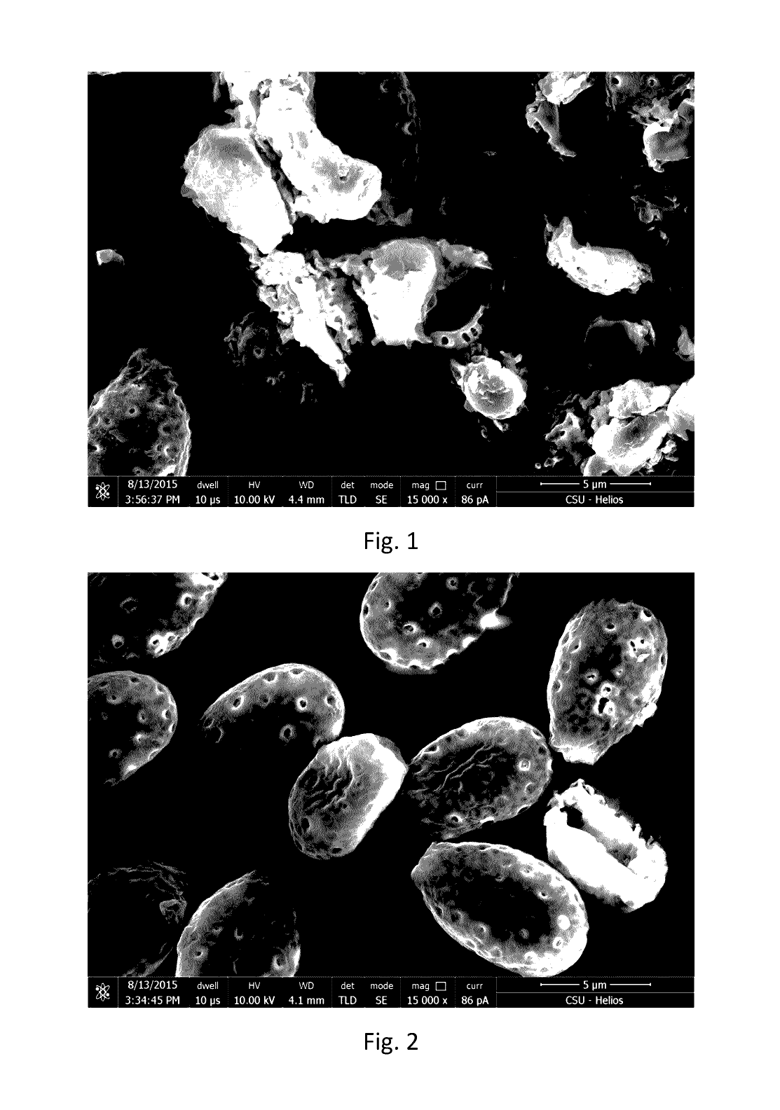

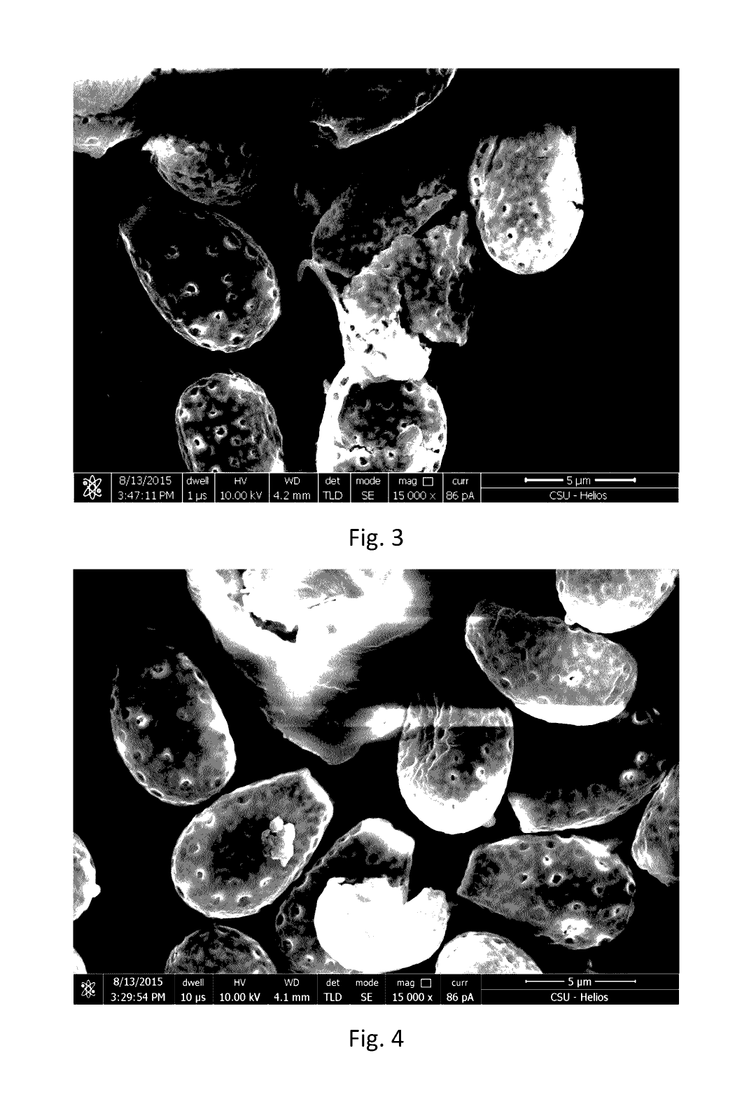

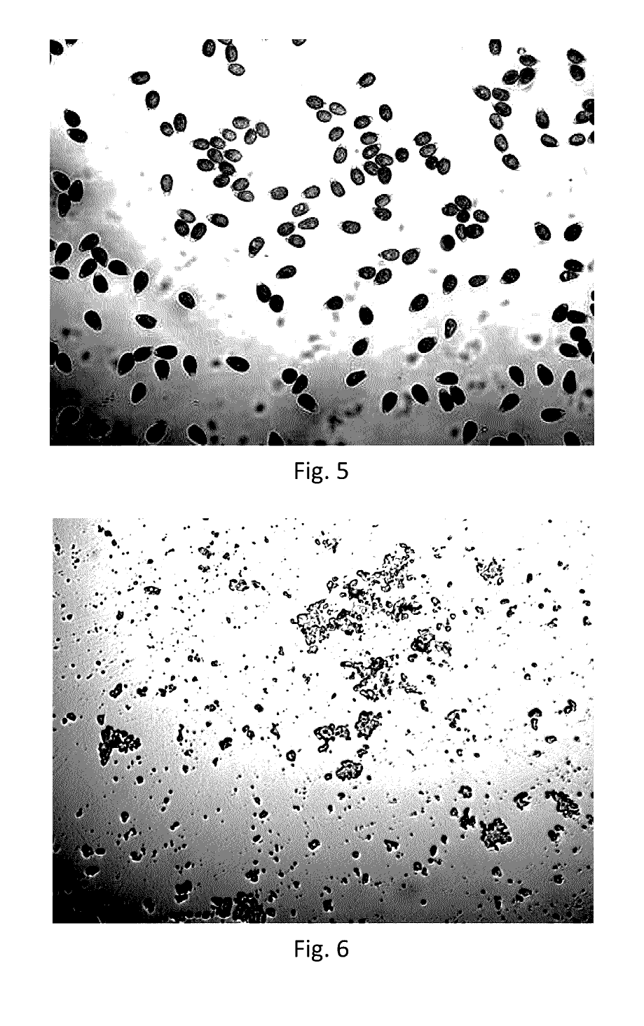

Results of the electron microscopic analyses are as shown in FIG. 1 through FIG. 6. FIG. 1 shows an electron microscopic image of Ganoderma spores after 30 min of decoction; FIG. 2 shows an electron microscopic image of Ganoderma spores after 10 min of decoction; FIG. 3 shows an electron microscopic image of Ganoderma spores after 20 min of decoction; FIG. 4 shows an electron microscopic image of Ganoderma spores after 20 min of microwaving; FIG. 5 shows an optical microscopic image of sporoderm-intact Ganoderma spores under 400× magnification; and FIG. 6 shows an optical microscopic image of sporoderm-broken Ganoderma spores under 400× magnification.

The method for breaking sporoderm of Ganoderma spore powder and the products obtained by the same provided by the present invention will be described below in detail in combination with specific embodiments. It is to be noted that 1 part by weight referred hereafter is 1 g and 1 part by volume referred hereafter is 1 mL.

### Embodiment 1

This embodiment provides a method for breaking sporoderm of Ganoderma spore powder, which includes the following steps:

putting 5 parts by weight of Ganoderma spore powder into 100 parts by volume of distilled water; stirring fully to form a mixture; slowly heating the mixture to 63° C. and maintaining at 63° C. for 45 min to obtain a sporoderm-broken spore powder solution; optionally, filtering the sporoderm-broken spore powder solution; pooling and drying the filtrates obtain dried Ganoderma spore powder extract.

### Embodiment 2

This embodiment provides a method for breaking sporoderm of Ganoderma spore powder, which includes the following steps:

putting 7 parts by weight of Ganoderma spore powder into 70 parts by volume of distilled water; stirring fully to form a mixture, slowly heating the mixture to 96° C. and maintaining at 96° C. for 20 min to obtain a primary sporoderm-broken spore powder solution; filtering the primary sporoderm-broken spore powder solution to obtain a primary filtrate and primary filtration residue;

adding 70 parts by volume of distilled water into the primary filtration residue; stirring fully; slowly heating to 100° C. and maintaining at 100° C. for 20 min to obtain a secondary sporoderm-broken spore powder solution; filtering the secondary sporoderm-broken spore powder solution to obtain a secondary filtrate; and pooling and drying the secondary filtrate and the primary filtrate to obtain a dried Ganoderma spore powder extract.

The Ganoderma spore powder in this embodiment is preferably Ganoderma spore powder not having been subjected to a sporoderm breaking treatment.

### Embodiment 3

This embodiment provides a method for breaking sporoderm of Ganoderma spore powder, which includes the following steps:

putting 10 parts by weight of Ganoderma spore powder into 60 parts by volume of distilled water; stirring fully to form a mixture; slowly heating the mixture to 78° C. and maintaining at 78° C. for 60 min to obtain a primary sporoderm-broken spore powder solution; filtering the primary sporoderm-broken spore solution to obtain a primary filtrate and a primary filtration residue;

adding 70 parts by volume of distilled water into the primary filtration residue; stirring fully; slowly heating to 90° C. and maintaining at 90° C. for 25 min to obtain a secondary sporoderm-broken spore powder solution; filtering the secondary sporoderm-broken spore powder solution to obtain a secondary filtrate; pooling and drying the secondary filtrate and the primary filtrate to obtain a dried Ganoderma spore powder extract.

### Embodiment 4

This embodiment provides a method for breaking sporoderm of Ganoderma spore powder, which includes the following steps:

putting 5 parts by weight of Ganoderma spore powder into 80 parts by volume of distilled water; stirring fully to form a mixture; slowly heating the mixture to 100° C. and maintaining at 100° C. for 15 min to obtain a primary sporoderm-broken spore powder solution; filtering the primary sporoderm-broken spore powder solution to obtain a primary filtrate and a primary filtration residue;

adding 80 parts by volume of distilled water into the primary filtration residue; stirring fully; slowly heating to 100° C. and maintaining at 100° C. for 15 min to obtain a secondary sporoderm-broken spore powder solution; filtering the secondary sporoderm-broken spore powder solution to obtain a secondary filtrate and a secondary filtration residue;

adding 80 parts by volume of distilled water into the secondary filtration residue; stirring fully; slowly heating to 100° C. and maintaining at 100° C. for 15 min to obtain a tertiary sporoderm-broken spore powder solution; filtering the tertiary sporoderm-broken spore powder solution to obtain a tertiary filtrate; and pooling and drying the primary filtrate, the secondary filtrate and the tertiary filtrate to obtain a dried Ganoderma spore powder extract.

### Embodiment 5

This embodiment provides a method for breaking sporoderm of Ganoderma spore powder, which includes the following steps:

putting 5 parts by weight of Ganoderma spore powder into 100 parts by volume of distilled water; stirring fully to form a mixture; slowly heating the mixture to 100° C. and maintaining at 100° C. for 15 min to obtain a sporoderm-broken spore powder solution; optionally, filtering the sporoderm-broken spore powder solution; and pooling and drying the filtrate to obtain a dried Ganoderma spore powder extract.

The Ganoderma spore powder in this embodiment is preferably Ganoderma spore powder not having been subjected to a sporoderm breaking treatment.

### Embodiment 6

This embodiment provides a steamed bun prepared by the following method:

Mixing the dried Gganoderma spore powder extract obtained according to any one of Embodiments 1 to 5 with a proper amount of flour and yeast; kneading the mixture into a dough; fermenting, shaping, and proofing the dough; and steaming the dough in a steamer until the dough is cooked.

### Embodiment 7

This embodiment provides a beverage prepared by the following method:

Adding a proper amount of water into the dried Ganoderma spore powder extract obtained according to any one of Embodiments 1 to 5 to obtain the beverage.

### Embodiment 8

A beverage prepared from the sporoderm-broken spore powder solution directly obtained by the sporoderm-breaking method according to Embodiments 1 to 5 is provided.

### Embodiment 9

Ganoderma spore powder together with raw materials for porridge, such as rice or millet, is put into 60 to 100 parts by volume of water and stirred fully. The mixture is slowly heated to a temperature ranging between 63 and 100° C., and the temperature is maintained for 15 to 60 min to obtain a spore powder porridge.

### Embodiment 10

The sporoderm-broken spore powder solution directly obtained by the sporoderm-breaking method according to Embodiments 1 to 5 is added with flour and kneaded into a dough to obtain a steamed bun, cake, bread, cookie, or other staple foods and desserts.

### Embodiment 11

The sporoderm-broken spore powder solution directly obtained by the sporoderm-breaking method according to Embodiments 1 to 5 is added into a carbonated beverage, fruit-vegetable juice beverage, tea beverage, milk beverage, coffee beverage, and alcoholic beverage to obtain a sporoderm-broken spore powder-containing carbonated beverage, fruit-vegetable juice beverage, tea beverage, milk beverage, coffee beverage and alcoholic beverage.

Apparently, the foregoing embodiments are merely examples for clarification, and not intended to limit the implementation of the present invention. A person of ordinary skill in the art would know to make changes or variations of other forms on the basis of the foregoing description. All implementations are unnecessarily and cannot be enumerated herein. All apparent changes or variations derived from the foregoing description shall fall into the protection scope of the present invention.

What is claimed is:

1. A method for breaking sporoderm of Ganoderma spore powder, comprising the steps of:
(S1) putting 5 to 10 parts by weight of Ganoderma spore powder into 60 to 100 parts by volume of water;
(S2) stirring fully to form a mixture; and
(S3) performing a sporoderm-breaking treatment on the mixture to obtain a sporoderm-broken spore powder solution,
wherein the sporoderm-breaking treatment comprises slowly heating the mixture to a temperature ranging between 63 and 100° C.; and maintaining at the temperature for 15 to 60 min.

2. The method for breaking sporoderm of Ganoderma spore powder according to claim 1, wherein the sporoderm-breaking treatment further comprises slowly heating the mixture to a temperature ranging between 96 and 100° C.; and maintaining at the temperature for 15 to 20 min.

3. The method for breaking sporoderm of Ganoderma spore powder according to claim 1, wherein steps (S1) to (S3) further comprise the steps of:
(S1a) putting 5 parts by weight of the Ganoderma spore powder into 100 parts by volume of distilled water;
(S2a) stirring fully to form the mixture; and
(S3a) slowly heating the mixture to 63° C. and maintaining at 63° C. for 45 min.

4. The method for breaking sporoderm of ganoderma spore powder according to claim 1, wherein steps (S1) to (S3) further comprise the steps of:
(S1b) putting 7 parts by weight of the Ganoderma spore powder into 70 parts by volume of distilled water;
(S2b) stirring fully to form the mixture; and
(S3b) slowly heating the mixture to 96° C. and maintaining at 96° C. for 20 min.

5. The method for breaking sporoderm of Ganoderma spore powder according to claim 1, wherein steps (S1) to (S3) further comprise the steps of:
(S1c) putting 10 parts by weight of the Ganoderma spore powder into 60 parts by volume of distilled water;
(S2c) stirring fully to form the mixture; and
(S3c) slowly heating the mixture to 78° C. and maintaining at 78° C. for 60 min.

6. The method for breaking sporoderm of Ganoderma spore powder according to claim 1, wherein steps (S1) to (S3) further comprise the steps of:
(S1d) putting 5 parts by weight of the Ganoderma spore powder into 80 parts by volume of distilled water;
(S2d) stirring fully to form the mixture; and
(S3d) slowly heating the mixture to 100° C. and maintaining at 100° C. for 15 min.

7. A sporoderm-broken spore powder solution obtained by the steps of:
(S1) putting 5 to 10 parts by weight of Ganoderma spore powder into 60 to 100 parts by volume of water;
(S2) stirring fully to form a mixture; and
(S3) performing a sporoderm-breaking treatment on the mixture to obtain a sporoderm-broken spore powder solution,
wherein the sporoderm-breaking treatment comprises slowly heating the mixture to a temperature ranging between 63 and 100° C.; and
maintaining at the temperature for 15 to 60 min.

8. A dried Ganoderma spore powder extract obtained by the steps of:
(S1) putting 5 to 10 parts by weight of Ganoderma spore powder into 60 to 100 parts by volume of water;
(S2) stirring fully to form a mixture;
(S3) performing a sporoderm-breaking treatment on the mixture to obtain a sporoderm-broken spore powder solution;
(S4) filtering the sporoderm-broken spore powder solution to obtain a plurality of filtrates; and
(S5) pooling and drying the filtrates to obtain a dried Ganoderma spore powder extract,
wherein the sporoderm-breaking treatment comprises slowly heating the mixture to a temperature ranging between 63 and 100° C.; and maintaining at the temperature for 15 to 60 min.

9. The dried Ganoderma spore powder extract according to claim 8, wherein steps (S4) to (S5) further comprise the steps of:
(S4a) filtering the primary sporoderm-broken spore powder solution to obtain a primary filtrate and a primary filtration residue;
(S5a) adding 70 parts by volume of distilled water into the primary filtration residue, stirring fully, slowly heating to 100° C. and maintaining at 100° C. for 20 min to obtain a secondary sporoderm-broken spore powder solution;
(S6a) filtering the secondary sporoderm-broken spore powder solution to obtain a secondary filtrate; and
(S7a) pooling and drying the secondary filtrate and the primary filtrate to obtain the dried ganoderma spore powder extract.

10. The dried Ganoderma spore powder extract according to claim 8, wherein steps (S4) to (S5) further comprise the steps of:
(S4b) filtering the primary sporoderm-broken spore powder solution to obtain a primary filtrate and a primary filtration residue;
(S5b) adding 70 parts by volume of distilled water into the primary filtration residue, stirring fully, slowly heating to 90° C. and maintaining at 90° C. for 25 min to obtain a secondary sporoderm-broken spore powder solution;
(S6b) filtering the secondary sporoderm-broken spore powder solution to obtain a secondary filtrate; and
(S7b) pooling and drying the secondary filtrate and the primary filtrate to obtain the dried Ganoderma spore powder extract.

11. The dried Ganoderma spore powder extract according to claim 8, wherein steps (S4) to (S5) further comprise the steps of:
(S4c) filtering the primary sporoderm-broken spore powder solution to obtain a primary filtrate and a primary filtration residue;
(S5c) adding 80 parts by volume of distilled water into the primary filtration residue, stirring fully, slowly heating to 100° C. and maintaining at 100° C. for 15 min to obtain a secondary sporoderm-broken spore powder solution;
(S6c) filtering the secondary sporoderm-broken spore powder solution to obtain a secondary filtrate and a secondary filtration residue;
(S7c) adding 80 parts by volume of distilled water into the secondary filtration residue, stirring fully, slowly heating to 100° C. and maintaining at 100° C. for 15 min to obtain a tertiary sporoderm-broken spore powder solution;
(S8c) filtering the tertiary sporoderm-broken spore powder solution to obtain a tertiary filtrate; and
(S9c) pooling and drying the primary filtrate, the secondary filtrate and the tertiary filtrate to obtain the dried Ganoderma spore powder extract.

12. A food, comprising a sporoderm-broken spore powder solution obtained by the steps of:
(S1) putting 5 to 10 parts by weight of Ganoderma spore powder into 60 to 100 parts by volume of water;
(S2) stirring fully to form a mixture; and
(S3) performing a sporoderm-breaking treatment on the mixture to obtain a sporoderm-broken spore powder solution,
wherein the sporoderm-breaking treatment comprises slowly heating the mixture to a temperature ranging between 63 and 100° C.; and maintaining at the temperature for 15 to 60 min.

13. The food according to claim 12, wherein the food comprises: porridge; steamed bun; cake; bread; cookie; staple foods; and desserts.

14. The food according to claim 12, comprising a beverage containing the sporoderm-broken spore powder solution, wherein the beverage comprises: carbonated beverage; fruit-vegetable juice beverage; tea beverage; milk beverage; coffee beverage; and alcoholic beverage.

15. A food, comprising a dried Ganoderma spore powder extract obtained by the steps of:
(S1) putting 5 to 10 parts by weight of Ganoderma spore powder into 60 to 100 parts by volume of water;
(S2) stirring fully to form a mixture;
(S3) performing a sporoderm-breaking treatment on the mixture to obtain a sporoderm-broken spore powder solution;
(S4) filtering the sporoderm-broken spore powder solution to obtain a plurality of filtrates; and
(S5) pooling and drying the filtrates to obtain a dried Ganoderma spore powder extract,
wherein the sporoderm-breaking treatment comprises slowly heating the mixture to a temperature ranging between 63 and 100° C.; and maintaining at the temperature for 15 to 60 min.

16. The food according to claim 15, wherein the food comprises: porridge; steamed bun; cake; bread; cookie; staple foods; and desserts.

17. The food according to claim 15, comprising a beverage containing the dried Ganoderma spore powder extract, wherein the beverage comprises: carbonated beverage; fruit-vegetable juice beverage; tea beverage; milk beverage; coffee beverage; and alcoholic beverage.
